(19) United States
(12) Patent Application Publication
(10) Pub. No.: US 2009/0306091 A1
(43) Pub. Date: Dec. 10, 2009

(54) ANTIMALARIAL THERAPY USING A COMBINATION OF SYNTHETIC ARTEMISININ DERIVATIVE AND BISQUINOLINE DERIVATIVE

(76) Inventors: Vyas Madhavrao Shingatgeri, Gurgaon (IN); Venkatesha Udupa, Bangalore (IN)

Correspondence Address: Ranbaxy Inc., Intellectual Property Department, 600 College Road East, PRINCETON, NJ, 08540, US

(21) Appl. No.: 12/300,921

(22) PCT Filed: May 17, 2007
(86) PCT No.: PCT/IB07/51898

§ 371 Date: Feb. 26, 2009

(30) Foreign Application Priority Data
May 17, 2006 (IN) 1218/DEL/2006

(51) Int. Cl. A61K 31/496; A61K 31/47; A61P 33/06

(52) U.S. Cl. 514/252.11; 514/314

(57) ABSTRACT

The technical field of the present invention relates to antimalarial therapy using a synthetic artemisinin derivative and bisquinoline derivative.

### FIELD OF THE INVENTION

The technical field of the present invention relates to antimalarial therapy using a synthetic artemisinin derivative and bisquinoline derivative.

### BACKGROUND OF THE INVENTION

Malaria is a serious and sometimes fatal disease caused by a parasite; Plasmodium falciparum, Plasmodium vivax, Plasmodium Ovale, and Plasmodium malariae. Patients with malaria typically are very sick with high fevers, shaking chills, and flu-like illness, whereas infection with Plasmodium falciparum, if not promptly treated, may be even fatal.

Malaria continues to be one of the most important and devastating infectious disease in developing areas of the world. Worldwide, over 40% of the population lives in areas where malaria transmission occurs (i.e., parts of Africa, Asia, the Middle East, Central and South America, Hispaniola and Oceania). It is estimated that 300-500 million cases of malaria occur each year resulting in 750,000-2 million deaths. According to an estimate, every 30 seconds in Africa, a child dies of malaria.

In principle, malaria is a curable and preventable disease. Preventive measures focus mainly on the avoidance of mosquito bites and vector control measures to reduce mosquito transmission. WHO recommends that, treatment should start immediately within 24 hours of appearance of symptoms of infection. Drugs available for treatment include chloroquine, sulphadoxine-pyrimethamine, mefloquine, atovaquone-proguanil, and quinine. The malaria situation is still worsening because the parasite has become resistant to most of the available treatment options.

One of the most potent and effective antimalarial drugs, especially in combination with drugs of other classes, includes artemisinin and its derivatives, extracted from sweet wormwood or Chinese wormwood—Artemisia annua. Perceiving the potential threat of malarial parasite developing resistance towards, otherwise very potent, artemisinin class of drugs, WHO has called for an immediate halt to provision of single-drug artemisinin malaria pills (see press release of 19 Jan. 2006 at www.who.int/mediacentre/news/release/2006/pr02). The combination of artemisinin derivatives available, include artemether and lumefantrine, artesunate and amodiaquine, artesunate and mefloquine, artesunate and sulfadoxine/pyrimethamine, dihydroartemisinin and piperaquine phosphate.

The available artemisinin based drugs are derived from natural sources, and the huge demand is far from being met with the current production capacity. In November 2004, WHO announced a shortage of artemisinin based combination, which is expected to continue in the following years.

Combination therapy is broadly accepted in the treatment of several diseases, such as TB, HIV infection, and cancer. In the case of malaria combination therapy has been applied since around 1990. However, this strategy is being hampered because the Plasmodium parasite has developed resistance, as a result of monotherapy, to certain components of currently applied combination drugs.

The idea behind combination therapy is that one of the drug would rapidly kill most of the Plasmodium parasites, and those that survive are subsequently killed by a high concentration of the companion drug. Combination therapy is thus expected to retard the development of resistance, improve efficacy by lowering recrudescence rate, provide synergistic effect, and increase exposure of the parasite to the drugs.

Selection of these combinations as an antimalarial therapy is thus based on certain attributes. Synthetic artemisinin derivatives exhibit their action by their reaction with the iron in free heme molecules in the malaria parasite with the generation of free radicals leading to cellular destruction. On the other hand bisquinoline derivatives interfere with the detoxification of haemin in the digestive vacuole of the parasite to non-toxic malaria pigment, so that haemin can generate free radicals and membrane damage follows. The unrelated mode of action of the two drugs would provide improved therapy, and treatment against all stages of parasites including gametocytes. Additionally since, synthetic artemisinin derivatives are very efficacious and highly potent; these would thereby treat the symptoms quickly, exhibiting fast recovery rates.

Monotherapy with artemisinin class of drugs may cure the patient in 7 days. However, studies conducted on animals, revealed that synthetic artemisinin derivatives cure the animals within 3 days. Combination of synthetic artemisinin derivatives and bisquinoline derivatives would thus provide a short duration of treatment. Further, the published literature, as well as the preliminary studies conducted by us, revealed no negative pharmacological interaction between synthetic artemisinin derivatives and bisquinoline derivatives, thereby combining these two classes of drugs would be logical.

The synthetic artemisinin derivatives have a short duration of action (t_{1/2} few hours) whereas bisquinolines have a long duration of action (t_{1/2} few weeks), bisquinolines would thus be present in the circulation in sufficient concentration, even after 2 cycles exposure to synthetic artemisinin derivatives. This would ensure the killing of any parasites remaining after therapy. Further, the smaller t_{1/2} of synthetic artemisinin derivatives make them less vulnerable to development of resistance.

General toxicity studies conducted on animals as per ICH guidelines in a GLP compliant laboratory, confirm the tolerability of the synthetic artemisinin derivatives over as wide dose range. Further, embryo-fetal assessment studies conducted on animals revealed that synthetic artemisinin derivative has comparatively lower risk than artesunate. Similarly, tolerability and toxicity studies conducted on animals as per OECD guidelines in a GLP compliant laboratory, confirmed the tolerability and well accepted toxicity profile of bisquinoline derivative. Further, toxicity studies conducted on animals using combination of synthetic artemisinin derivatives and bisquinoline derivatives, revealed an additive toxicity profile. The drugs thus do not significantly increase the toxicity of each other when used in combination.

Finally, synthetic artemisinin derivatives and bisquinoline derivatives may be synthesized in GLP compliant facility using validated synthetic processes, and thereby an uninterrupted and economic supply of the bulk drugs can be maintained, as per demand.

Thus, there exists a need for the development of novel combination therapy based on synthetic artemisinin derivatives.

### SUMMARY OF THE INVENTION

Hence, in one of the general aspect there is provided, antimalarial therapy using a combination of synthetic artemisinin derivative and bisquinoline derivative.

In another general aspect there is provided, antimalarial therapy using a combination of synthetic artemisinin derivative and piperaquine.

In another general aspect there is provided, antimalarial therapy using a combination of cis-adamantane-2-spiro-3′-8′-[[[(2′-amino-2′methylpropyl)amino]carbonyl]methyl]1′, 2′, 4′-trioxaspiro[4.5]decane and bisquinoline derivative.

In another general aspect there is provided, antimalarial therapy using a combination of cis-adamantane-2-spiro-3′-8′-[[[(2′-amino-2′methylpropyl)amino]carbonyl]methyl]1′, 2′, 4′-trioxaspiro[4.5]decane and piperaquine.

Based on the data compiled from published literature and promising results from preclinical studies, synthetic artemisinin derivatives and bisquinoline derivatives appear to satisfy the criteria for an ideal combination for antimalarial therapy.

Synthetic artemisinin derivatives include one or more of the various spiro and dispiro trioxolanes derivatives disclosed in US 2004/0186168, U.S. Pat. No. 6,486,199 and U.S. Pat. No. 6,825,230; particularly cis-adamantane-2-spiro-3′-8′-[[[(2′-amino-2′methylpropyl)amino]carbonyl]methyl]1′,2′,4′-trioxaspiro[4.5]decane. These trioxolanes are relatively sterically hindered on at least one side of the trioxolane heterocycle, which provides better in vivo activity, especially with respect to oral administration. Particularly, spiro and dispiro 1,2,4-trioxolanes derivatives possess excellent potency and efficacy against Plasmodium parasites, and a lower degree of neurotoxicity. Synthetic artemisinin derivatives includes free form of the compounds referred to herein as well as their pharmaceutically acceptable salts, solvates, esters, enantiomers, diastereomers, polymorphs, metabolites, prodrugs and analogues.

Bisquinoline derivatives are compounds with two quinoline nuclei bound by a covalent aliphatic or aromatic link. Several of these compounds were identified as promising antimalarial candidates and include Hydroxypiperaquine, dichlorquinazine, 1,4-bis(7-chloro-4-quinolylamino)piperazine, and piperaquine, particularly piperaquine. Bisquinolines includes free form of the compounds as well as their pharmaceutically acceptable salts, solvates, esters, enantiomers, diastereomers, polymorphs, metabolites, prodrugs and analogues.

Therapeutically effective amounts of synthetic artemisinin derivatives and bisquinoline derivatives may be combined with one or more pharmaceutically inert excipients and processed into in to suitable single or separate pharmaceutical compositions. Combination antimalarial therapy may be achieved by administering single pharmaceutical compositions or separate pharmaceutical compositions, simultaneously or sequentially, in a dosage regimen, as appropriate. Route of administration of the pharmaceutical compositions may be one or more of any of the possible routes, based on the patient's condition and other critical parameters, and includes oral, parenteral, rectal, vaginal, and transmucosal.

While the present invention has been described in terms of its specific embodiments, certain modifications and equivalents will be apparent to those skilled in the art and are included within the scope of the present invention.

### EXAMPLE

### Embryo-Fetal Studies with cis-Adamantane-2-spiro-3′-8′-[[[(2′-amino-2′methylpropyl)amino]carbonyl]methyl]1′,2′,4′-trioxaspiro[4.5]decane hydrogen (Oral Administration)

Embryo-fetal assessment studies were conducted in rats and rabbits with oral administration of cis-adamantane-2-spiro-3′-8′-[[[(2′-amino-2′methylpropyl)amino]carbonyl methyl]1″,2′,4′-trioxaspiro[4.5]decane at 10, 30 and 90 mg/kg/day dose level as per ICH guideline in a GLP Compliant Laboratory. There was no accumulation after repeated dosing.

### Studies on Female Rats

All rats survived until scheduled Caesarean section, and no test item-related clinical signs, signs of discomfort and gross changes were noted.

At 10 and 30 mg/kg/day dose level, no changes in food consumption, mean body weight, reproductive parameters, and fetus weight were noticed.

At 90 mg/kg/day dose level, reduction in mean food consumption (−10.7% compared with the vehicle control), body weight gain (57 g compared with 74 g in the vehicle control), post-implantation loss (10/22 females), litter size (8.2 fetuses per dam compared with 13.6 in the vehicle control) and fetal weight (4.1 g compared with 4.8 g in the vehicle control) were noticed. Further, abnormal findings viz., interventricular septal defect (5 fetuses), aortic arch dilated (6 fetuses), ductus arteriosus and/or pulmonary trunk narrowed (5 fetuses), spleen reduced in size (4 fetuses) were noticed.

No test item-related effects were noted on fetal sex ratios, at fresh external examination or at skeletal examination at all dose levels. Further, there was no accumulation after repeated dosing.

Based on these results the NOEL (no observed effect level) for maternal organisms and reproductive parameters was considered to be 30 mg/kg body weight/day With respect to fetal organisms the NOEL (no observed effect level) was considered to be 10 mg/kg body weight/day.

### Studies on Female Rabbits

At 90 mg/kg/day dose level, labored respiration was noted for four females and one female was found dead on day 19 pc, reduction in food consumption (−27.3% compared with the vehicle control), mean body weight development (+0.1% compared with +2.4% in the vehicle control), fetal weight (31.5 g compared with 32.7 g in the vehicle control),

At 10 and 30 mg/kg/day dose level, no mortality/clinical signs, changes in mean body weight and post-implantation loss.

At 30 mg/kg body weight/day, slight reduction in food consumption was noticed.

No macroscopic findings and fetal anomalies were noticed in any dose levels. Further, there was no accumulation after repeated dosing.

Based on these results the NOEL (no observed effect level) for maternal organisms was considered to be 10 mg/kg body weight/day, and NOEL for fetal organisms was considered to be 30 mg/kg body weight/day.

The studies did not reveal any teratogenic potential up to and including a dosage of 90 mg/kg body weight/day.

On the contrary, reports (Clark et al., 2004) on embryofetal assessment in rat with artesunate revealed post implantation loss, ventricular septal defect and several skeletal malformations at as low as 6 mg/kg/day, and in rabbit, post implantation loss, cleft palate, aortic arch dilatation, pulmonary trunk dilatation and several skeletal malformations at 12 mg/kg/day.

The above studies clearly conclude that embryofetal risk with cis-adamantane-2-spiro-3′-8′-[[[(2′-amino-2′methylpropyl)amino]carbonyl methyl]1′,2′,4′-trioxaspiro[4.5]decane—is comparatively better than Artesunate.

### REFERENCE

Clark R L, White T K, A Clode S, Gaunt I, Winstanley P and Ward S A. (2004). Developmental toxicity of Artesunate and an Artesunate combination in rat and rabbit. Birth defects Res B Dev Reprod Toxicol. Dec. 71(6); 380-394.

We claim:

1. Antimalarial therapy using a combination of synthetic artemisinin derivative and bisquinoline derivative.

2. Antimalarial therapy according to claim 1 wherein the bisquinoline derivative is piperaquine.

3. Antimalarial therapy according to claim 1 wherein the artemisinin derivative is cis-adamantane-2-spiro-3′-8′-[[[(2′-amino-2′methylpropyl)amino]carbonyl]methyl]1′, 2′, 4′-trioxaspiro[4.5]decane.

4. Antimalarial therapy according to claim 1 wherein the artemisinin derivative is cis-adamantane-2-spiro-3′-8′-[[[(2′-amino-2′methylpropyl)amino]carbonyl]methyl]1′, 2′, 4′-trioxaspiro[4.5]decane and bisquinoline derivative is piperaquine.

5. Antimalarial therapy according to any of the preceding claims wherein combination antimalarial therapy may be achieved by administering single pharmaceutical compositions or separate pharmaceutical compositions, simultaneously or sequentially.

6. Antimalarial therapy according to claim 5 wherein route of administration of the pharmaceutical compositions may be one or more of oral, parenteral, rectal, vaginal, and transmucosal routes.
